(19) United States
(12) Patent Application Publication
(10) Pub. No.: US 2009/0163943 A1
(43) Pub. Date: Jun. 25, 2009

(54) MULTI-PURPOSE TOOL FOR MINOR SURGERY

(76) Inventors: Brian J. Cavanaugh, Hingham, MA (US); James J. A. Cavanaugh, Osterville, MA (US)

Correspondence Address: RAYMOND R. MOSER JR., ESQ.;MOSER IP LAW GROUP, 1030 BROAD STREET, 2ND FLOOR, SHREWSBURY, NJ, 07702, US

(21) Appl. No.: 12/339,843

(22) Filed: Dec. 19, 2008

Related U.S. Application Data
(60) Provisional application No. 61/008,371, filed on Dec. 20, 2007.

(51) Int. Cl. A61B 17/22

(52) U.S. Cl. 606/180

(57) ABSTRACT

In one embodiment, there is multi-purpose tool comprising: a supporting body adapted to receive and support a bit; a handle attached to the supporting body; a view controlling means coupled to, or adapted to couple to, the supporting body for controlling the view of a viewing device; and one or more of the following control means: (i) a rotatory controlling means coupled to the supporting body for controlling the rotational movement of a bit; and (ii) a pincer controlling means coupled to the supporting body for controlling pincing movement of the bit. In one embodiment, (i) pertains. In another embodiment, (ii) pertains. In another embodiment, (i) and (ii) pertains. In one embodiment, the means of (i) and (ii) share a common actuator and, in some embodiments, additional shared mechanical parts.

This application claims benefit of U.S. provisional patent application Ser. No. 61/008,371, filed Dec. 20, 2007, which is herein incorporated by reference.

### BACKGROUND

Physicians and other medical practitioners face multiple challenges when attempting to carry out a medical procedure within a blind cavity of a patient. The presently available medical instruments that are used to carry out such procedures do not adequately address these challenges. Most of the medical instruments that are used within the ear canal, oral cavity (mouth), throat, vagina or rectum require secondary lighting, significant manual manipulation and an interchange between one or more instruments.

To better understand the medical practitioner's challenge it is necessary to describe one of the blind cavities and the associated procedures performed therein. The human ear, for example, is a complex tripartite sensory end organ that encompasses hearing and balance functions. The external portion of the ear connects to the inner ear through a short passage way, the external auditory canal, and ends at a thin, highly sensitive tissue, the tympanic membrane, that occludes the interior end of the canal and connects to the three (3) small bones of the middle ear known as the ossicles. Sound vibration of the tympanic membrane is transmitted through the ossicles to the inner ear complex comprised of three (3) distinct parts: the central vestibules, the semicircular canals and the cochlea. The semicircular canals control balance and motion while the cochlea, a canal wound around a central post like the shell of a snail, contains the hearing apparatus or the Organ of Corti.

The direction of the external canal, a short, one-and-one-quarter inch tube, runs inward, forward, and downward and ends at the tympanic membrane creating a blind pouch that can vary significantly in length, diameter and direction.

The external auditory canal is an active participant in the sense of hearing and its integrity must be maintained to support its function. The canal is fibromembranous and is coated on its interior with a thin layer of tissue containing hairs, secreting glands, and sensory cells. Each of these components of the canal's lining is regularly shed or secreted into the canal lumen as cells, hairs, and a waxy substance called cerumen. The auditory canal is open externally to the environment and therefore is susceptible to injury, disease, and/or penetration, all of which have an impact on hearing function and the health of the auditory apparatus.

Since the secretion and shedding of various elements of the canal lining are physiological events, accumulation of cerumen, or wax, in the canal occurs normally but in varying amounts. For most individuals regular washing keeps the auditory canal open. In as many as 15% of children and adults, particularly in older adults, the configuration of the canal and the loss of moisture in the lining of the canal can lead to excessive secretion and plugging. When cerumen plugs occlude more than 50% of the diameter of the canal, hearing, particularly high frequency hearing, is impaired.

There is critical demand for a surgical instrument with universal capabilities to safely and efficiently resolve existing problems of blockage in the auditory canal due to the following factors:
(1) There is a significant and possibly increasing percentage of the population who have impacted cerumen;
(2) There is no appropriate instrument available to manage the problem of blockage;
ii) (3) There exists a trend in medical training toward specialization which limits the number of general physicians available to carry out such procedures; and

There is less time allowed per patient visit to resolve such problems due to insurance constraints.

Therefore, there exists a need to provide a surgical instrument that has wide applicability to be used not only within the ear canal, but also within other blind cavities. It must be safe and easy to use, able to illuminate and magnify the area of concern, and be easily adaptable for use in the general physician's or specialist's office setting.

The present invention addresses the multiple challenges presented when working in blind cavities, such as the ear canal, oral cavity (mouth), throat, vaginal and rectal canals when performing a variety of functions such as an examination and diagnosis, cleaning, tissue sampling for culture or biopsy, all of which require lighting, ease of use, direct vision and the need to interchange quickly and easily between a variety of tools.

There exist common features within the body where blind cavities are found. The problems, from a disease standpoint, represent foreign bodies, infection, tumors, and local manifestations of disease which are difficult to examine, often go unrecognized, and present technical hurdles for diagnosis and treatment. The architecture of the cavity poses problems which must be overcome in the skilled management of the malady. This is true for the ear canal, throat, rectum, or vagina for similar reasons. The availability of an instrument that can be skillfully applied with one hand utilizing a wide variety of operational bits for the required manipulation in a magnified and lighted surgical field which can be visually projected on an adjacent external monitor is a significant advantage.

As discussed above, one application for the present invention is a surgical tool for cleaning of the ear canal cerumen and managing the attending disability. In particular, the present invention addresses the multiple aspects of the physiological accumulation of secretions and cellular debris in the external auditory canal that causes blockage and the sequelae that follows.

In addition to wax accumulation, a wide variety of other abnormalities of the canal, the tympanic membrane, and/or the middle ear are not seen or are imperfectly diagnosed because of a combination of events:
(1) The configuration of the tube makes it difficult to view abnormalities;
   (2) The blockage increases the problem and makes it difficult for most medical practitioners to clear the wax and/or view the innermost areas of the canal, the tympanic membrane and what lies within; and
   (3) The resolution of (1) and (2) is time consuming for a busy office or practice resulting in missed diagnosis and imperfect resolution of problems

Removal of ear wax up to this date has been an ongoing problem for most physicians using inadequate tools that can cause pain, bleeding, or can damage the delicate membrane at the base of the canal. Use of suction or water pressure in the canal to clear wax can be painful and can rupture the tympanic membrane. As a multifunctional surgical instrument, this tool simplifies the surgical placement of ventilation tubes in the tympanic membranes of both children and adults with chronic otitis media.

### SUMMARY OF THE INVENTION

In one embodiment, the present invention is a multi-purpose tool that is, for example, comprised of two independent components: i) a multi-purpose hand-held device with a removable viewing device (camera), such as an endoscope, and electronics and operable bits (such as snap-in or non-snap-on bits); and ii) an adjustable monitor suspended from an adjustable boom which contains an image processor, and may be used with an input devices such as a keyboard, mouse or other. Both major components are each designed to be operated with just one hand. In all embodiments, interconnections between components may be hardwired or wireless. The multi-purpose hand-held device may be used with a display device known in the art or the display device may be the adjustable monitor suspended from the adjustable boom component indicated above in subsection ii.

In one embodiment, there is multi-purpose tool comprising: a supporting body adapted to receive and support a bit; a handle attached to the supporting body; a view controlling means coupled to, or adapted to couple to, the supporting body for controlling the view of a viewing device; and one or more of the following control means: (i) a rotatory controlling means coupled to the supporting body for controlling the rotational movement of a bit; and (ii) a pincer controlling means coupled to the supporting body for controlling pincing movement of the bit. In one embodiment, (i) pertains. In another embodiment, (ii) pertains. In another embodiment, (i) and (ii) pertains. In one embodiment, the means of (i) and (ii) share a common actuator and, in some embodiments, additional shared mechanical parts. Any tool herein can further comprise a viewing device coupled to, or adapted to couple to, the supporting body in a manner functionally engaged with the view controlling means. The view controlling means, if adapted to couple to the supporting body, can be incorporated in a viewing device adapted to couple to the supporting body.

In certain embodiments, the viewing device of the multi-purpose tool comprises a glass optic. In certain embodiments, the supporting body is adapted to receive a hollow bit, and where the a glass optic is shaped and positioned, or adapted to position, so that the hollow bit can be slipped over the glass optic to be received by the supporting body, such that when the bit is received the glass optic is positioned to visualize one or more areas that project along approximately a center axis of the tool bit.

In one embodiment, there is a multi-purpose tool comprising: a supporting body adapted to receive and support a bit; a handle attached to the supporting body; a rotatory controlling means coupled to the supporting body for controlling the rotational movement of a bit; and a pincer controlling means coupled to the supporting body for controlling pincing movement of the bit, and wherein one of the following pertains: (i) the supporting body is adapted to receive and support a viewing device; or (ii) the tool further comprises a viewing device.

The multi-purpose hand-held device is fundamentally a miniaturization of the user's eyes, fingers, and wrists to see and manipulate for the purpose of diagnosis and managements of maladies in a blind cavity. The hand-held device is, in certain embodiments, further designed to be held and operated by one hand only, gripping the tool in the crotch of the thumb and index finger. The ends of the thumb and index finger are left free to operate the three controls, with the middle finger available to steady the tool against some portion of the patient's body.

The multi-purpose hand-held device is, in certain embodiments, comprised of a removable viewing device, removable electronics and removable operable bits, supporting body, a handle, an optional handle extension, a view controlling means, a rotary controlling means, and a pincing controlling means. The handle is attached to the supporting body. A view controlling means can be used to control movement of a viewing device, which projects the image onto the adjustable monitor attached to the adjustable boom or a monitor as known in the art. A rotary controlling means can be used to control the rotational movement of a bit. The pincing controlling means is used to control the pincing or closing movement of the bit. Each controlling means instrument can be coupled to the supporting body.

The three controls that can be available on the hand-held device control the pincing and twisting actions of the bits with the third controlling the angle, position and/or focus of the camera (endoscope).

In another embodiment the present invention is a hand-held device comprising a supporting body, a handle, an internal viewing means, a view controlling means, a rotary controlling means, and a pincing controlling means. The handle is attached to the supporting body. All of the controlling means, i.e., view controlling means, rotary controlling means, and pincing controlling means can be coupled to the supporting body. The viewing means is internal to the supporting body. The view controlling means controls the view of the internal viewing means. The rotary controlling means is used to control the rotational movement of a bit. The pincing controlling means is used to control pincing movement of the bit.

The adjustable boom and adjustable monitor are designed for the other hand to operate. Fine positioning of the flat screen monitor or like to a vantage adjacent to where the image is projected from (the site of focus) can, for example, be achieved by lightly pulling on one of the omni-directional, multi-speed switches located, for example in a lower corner of the monitor. The switches can be located in both lower corners, and operated by the hand not operating the hand-held device. Non-motorized adjustments can also be used, such as utilizing springs, counterweights, adjustable friction at the joints, and the like as known in the art.

The adjustable monitor is designed to be adjusted with the same hand operating the monitor. A number of adjustable controls that are user associated to the processor adjusts contrast, hue, frequency (including infra red) to fine tune the view of the site of focus. In another embodiment, the adjustable monitor is a touch screen monitor.

In one application, the disclosed tool can be an effective cleaning tool for blockage of the external auditory canal. The tool is simple to manufacture, and is safe and easy to use. The tool can be easily adaptable in the general physician's office reducing time consumption currently associated with such procedures. In addition, the resultant design is ultimately a miniaturization and extension of the functional properties of the fingers, hand, forearm, and eyes. There are a number of alternative uses for the tools particularly in examining blind cavities other than the ear canal.

Further provided is a method of operating one of the multi-purpose tools by providing the tool; providing a power-assisted monitor comprising a means for moving the monitor with one hand; and operating the monitor with one hand and using the other hand to operate the tool by one or more of adjusting the view controlling means or the rotatory controlling means or the pincing controlling means. In operation, one hand is substantially dedicated (at least for a period of time) to operating the tool via the handle, while the other is free for other tasks including operating the monitor. Operating the monitor can comprise one or more of (a) adjusting via controls on the monitor light intensity provided by the tool, (b) adjusting via controls on the monitor wavelength of the provided light, or (c) adjusting via controls on the monitor the processing of camera output from the tool.

Also provided is a multi-purpose tool comprising: a supporting body adapted to receive and support a bit; a handle attached to the supporting body; a retaining mechanism for retaining a bit having a reglet, the mechanism comprising clip adapted to engage the reglet that is spring biased to the position for engaging the reglet but where the clip can be levered out of the engagement position.

While the invention is described herein by way of example using several embodiments and illustrative drawings, those skilled in art will recognize that the invention is not limited to the embodiments of the drawing or drawings described. It should be understood that the drawings and detailed description thereto are not intended to limit the invention to the particular form disclosed, but on the contrary, the invention is to cover all modifications, equivalents, and alternatives falling within the spirit and scope of the present invention as defined by the appended claims. As used throughout this application, the word “may” is used in a permissive sense (i.e., meaning having the potential to), rather than the mandatory sense (i.e., meaning must). Similarly, the words “include”, “including”, and “included” means including but not limited to. Further, the words “a” or “an” mean “at least one”, and the word “plurality” means one or more, unless otherwise mentioned.

### BRIEF DESCRIPTION OF THE DRAWINGS

FIG. 1 is a side view of an assembly comprising a multi-purpose hand-held device, a bit and a flexible camera like a fiber optic or like (endoscope) according to various embodiments of the present invention.

FIG. 2 illustrates a set of bits, for example the bits usable in the assembly of FIG. 1 and FIG. 7, according to various embodiments of the present invention.

FIG. 3 is a perspective partially cut-away view of the assembly of FIG. 1 illustrating the interior structure and mechanics of the multi-purpose hand-held device, a bit, and a flexible camera like a fiber optic or like (endoscope) according to various embodiments of the present invention.

FIG. 4 is a perspective view of the assembly of FIG. 1 in use according to various embodiments of the present invention.

FIG. 5 is a perspective partially cut-away view of an assembly illustrating the interior structure and mechanics of the multi-purpose hand-held device, bit, and central hard camera (endoscope), according to another embodiment of the present embodiment.

FIG. 6 is a perspective view of the assembly of FIG. 5 showing the central hard camera in use according to another embodiment of the present invention.

FIG. 7 is a perspective view of an assembly showing an offset hard camera (endoscope) with focus capabilities according to another embodiment of the present invention.

FIG. 8 is a perspective view of a display device according to another embodiment of the present invention which may be used in FIGS. 1-4, FIG. 5-6 and FIG. 7 illustrating a power assisted boom, which contains a processor, and an adjustable monitor.

FIG. 9 is a perspective view of an alternate quick change bit mechanism according to another embodiment of the present invention which could be used in lieu of the bit mechanism depicted in FIGS. 1, 3, 4, 5, 6 and 7.

FIG. 10 shows a detail section of a omni-directional-multi-speed switch assembly 1000 (see 894) that can control a power assisted monitor boom arm. So that the manner in which the above recited features of the present invention can be understood in detail, a more particular description of the invention, briefly summarized below, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

### DETAILED DESCRIPTION OF THE DRAWINGS

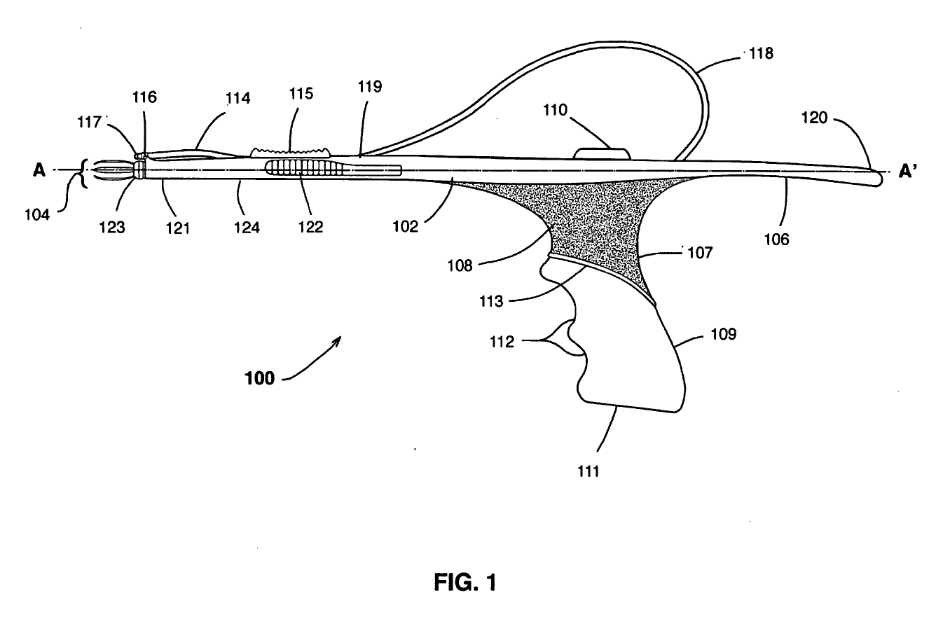

According to various embodiments, the present invention provides several methods of making the assembly 100, including but not limited to variations of the assembly 100 as shown and depicted in the FIGS. 1-4, FIGS. 5-6 and FIG. 7. As discussed, the method can comprise the supporting body, the handle, the handle extension, which is particularly optional, the viewing means (endoscope), which may be a flexible camera, like a fiber optic or like, centrally located hard camera, an offset camera or other known in the art, the view controlling means, the rotary controlling means, the pincing controlling means, and a handle adjustment and removal knob or variation thereof known in the art.

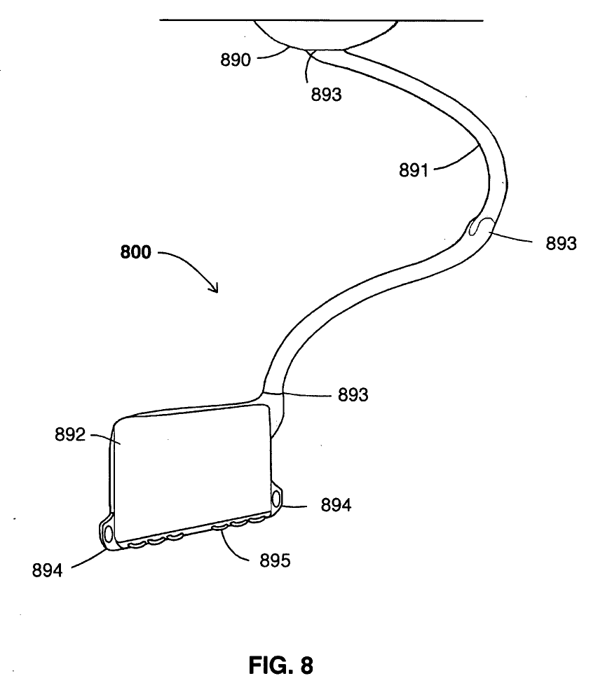

According to alternate embodiment, the present invention provides that the assembly, including but not limited to the variations of the assembly 100 as shown and depicted in the FIGS. 1-4, FIGS. 5-6 and FIG. 7, may be used with a display device that is an adjustable boom and adjustable monitor as shown in FIG. 8 or a display device known in the art.

FIG. 1 illustrates an assembly 100 according to various embodiments of the present invention. The assembly 100 comprises the multi-purpose hand-held device 102, bit 104, and viewing device 118. In one embodiment the tool is configured to be used for seeing and cleaning the outer ear canal. The multi-purpose hand-held device 102 may be held in the right hand or left hand. The bit 104 is received by the multi-purpose hand-held device 102 and the bit 104 is configured to operate within a body cavity or elsewhere.

Those skilled in the art will appreciate that, in certain uses of the present invention, the multi-purpose hand-held device 102 may comprise a view controlling means actuator 115 and rotary controlling means actuator 124. However, in alternative embodiments, the multi-purpose hand-held device 102 may comprise a view controlling means 115 and pincing controlling means actuator 122. Further, as depicted in FIG. 1, the multi-purpose hand-held device 102 may comprise a view controlling means 115, rotary controlling means actuator 123 and pincing controlling means actuator 122.

The multi-purpose hand-held device 102 can comprise a supporting body 106, a handle 107, a handle extension 109, of which its use is optional, for example dependent on the preference of the user. The supporting body 106 is a generally tubular body configured to receive the bit 104. Examples of the supporting body 106 may include metallic frames, non-metallic frames, and the like. The supporting body 106 has a proximal end 120 and a distal end 121, opposite to the proximal end. The proximal end 120 refers to a portion of the proximal end of the supporting body 106. Similarly, the distal end 121 refers to a portion of the distal end of the supporting body 106. Further, the supporting body 106 defines a body axis A-A′ as shown in the figure. When the multi-purpose hand-held device 102 is held before a body cavity, the distal end 121 is placed near the body cavity. The bit 104 is received by the distal end 121. However, in alternative embodiments, the bit 104 may be inserted through the proximal end 120. In alternative embodiments, the bit 104 may be inserted through an opening (not shown in figures) made into the supporting body 106, parallel to the body axis A-A′. The handle 107 can be, for example, attached to the supporting body 106 near the proximal end 120 with the help of the handle adjustment and removal knob 110. In the illustrated embodiment, the handle 107 which contains the tool's electronics is detachable from the supporting body 106. The handle 107 may be detached, for example, to facilitate autoclave cleaning of the remainder parts of the multi-purpose hand-held device 102. Further, the handle 107 may be replaced by handles of varied shapes and sizes to adjust to palm size of a user such as a male nurse or a female doctor. In other embodiments, the supporting body 106 and the handle 107 are configured such that the handle 107 may slide relative to the supporting body 106 while the handle adjustment and removal knob 110 is kept loose to accommodate the multi-purpose hand-held device 102 into hands having fingers of varying sizes. Once a desired position of the handle 107 is achieved, the handle adjustment and removal knob 110 may be tightened to restrict further relative motion of the handle 107 to the supporting body 106. The handle 107 is movable towards the proximal end 120 or towards the distal end 121. As a result, the handle 107 may be quickly adjusted according to the size of the user's hand. In alternative embodiments, the handle 107 may be rotated around the girth of the supporting body 106 for added comfort of the user and/or depending upon the application of the assembly 100. As it is known in the industry, the handle can be both removably attached and/or adjusted by alternate means.

In the illustrated embodiment, the handle 107 comprises a rubberized coating 108 that facilitates grip at the crotch of the hand while leaving free the distal ends of the fingers to operate the controls (view controller 115, pincing controlling means actuator 122, rotary controlling means actuator 124). It is believed that the other fingers such as the middle finger may function equally well for control of the tool.

In the illustrated embodiment, the handle 107 shows an added handle extension 109 for those who may need or desire further control in their grip. The handle extension 109 is easily attached and has finger indents 112 for the ring and pinky fingers for enhanced grip. The middle finger, typically the longest, is left free to steady the tool against the patient's body. The thumb and index finger are free to operate the multi-purpose hand-held device 102.

Further, in another embodiment, the handle 107 is composed of power cells and a transmitter unit (not shown) placed inside the handle 107. In one embodiment, the power cells may be rechargeable power cells where the multi-purpose hand held device 102 may be recharged by placing the handle 107 into a table top recharging unit using for example the charging contacts 113. In the embodiment where the handle extension 109 is attached to the handle 107, the table top recharging unit can, for example, rely on the charging contacts 111 which connect to contacts 113. The handle extension 109 may also contain auxiliary batteries. In an alternative embodiment, the base of the handle 107 may be connected using the charging contacts 113 to a transformer type electric plug for charging the rechargeable power cells or running the unit off the wall power via a thin set of wires. Alternatively, if the handle extension 109 is attached to the handle 107 then the charging contacts 111 will be used when connected to a transformer type electric plug for charging the rechargeable power cells or running the unit off the wall power via a thin set of wires. The images captured by the camera may be transmitted to a remote display device known in the art via hardwiring or transmission (such as Bluetooth) or the like.

In the embodiment shown in FIG. 1, the viewing device 118 is comprised of the micro lens 117, fiber element 114 and the remaining camera parts located, for example, inside the handle 107. This viewing device 118 as shown is a flexible endoscope like fiber optics, loops up out of the electronics case of handle 107 passing through a slot in the supporting body, then passing through the view controller channel 119 where it is releasably attached to the view controller 115. Near the distal end of the fiber element 114 is a piece of hardware that collects around the neck of the fiber cable (endoscope) that controls the angle of the micro lens via a small hinge 116. The image captured by the micro lens 117 can be, for example, transmitted in the handle 107 via Bluetooth or other wireless technology to the processor where operable controls at the adjustable monitor shown in FIG. 8 tailor the image through the processor as needed. Controls at the adjustable monitor allow for single-handed adjustment. Each of, for example, a half dozen mechanical- or touch screen-activated adjusters 895 (FIG. 8) are user-linked to any number of processor functions that adjust what is captured at the distal end of the camera and seen on the monitor. With the ability to adjust cavity light intensity and wavelength in both the visible and non-visible spectra, the resulting images with user adjustable superimposition mapping techniques, greatly enhance discovery of physical attributes and maladies beyond what is possible to identify by the naked eye.

In the current embodiment shown in FIG. 1, the fiber element 114 is removably coupled to the supporting body 106 via a hinge 116 in the vicinity of the distal end 121 as shown in FIG. 1. By moving the view controlling device 115, the fiber element 114 bows up and back down causing the micro lens 117 to change angle. The viewing device 118 is configured to access the interior of the body cavity. In an embodiment, the viewing device 118 may be coupled to the supporting body 106 in the vicinity of the proximal end 120. In an alternative embodiment, the viewing device 118 may be coupled to the supporting body 106 and positioned anywhere between the distal end 121 and proximal end 120. The removable light ring 123 is configured to provide light in the body cavity. The viewing device 118 is configured to transmit images of the interior of the body cavity to an adjustable monitor remote of the multi-purpose hand-held device 102.

According to alternate embodiment of the present invention, the viewing device 118 may be comprised of an internal or integral light source. When the multi-purpose hand-held device is used as an ear cleaning instrument, the light is configured to illuminate the interior of the auditory canal. The image micro lens 117 is adapted to take the image of the interior of the auditory canal and transmit the image through the fiber element 114 which is part of the viewing device 118. The image captured by the micro lens 117 may be transmitted by the viewing device 118 and displayed as an illuminated and magnified image by a display device (monitor) known in the art or the adjustable monitor as shown in FIG. 8. In alternative embodiments, the viewing device 118 may include an infra red (IR) camera for capturing IR images, heat sensors, depth gauges, high definition sensor and other such discovery tools, as known in the art. Different discovery tools operate to capture different wavelengths and the various wavelengths have various discovery properties. According to one embodiment, images or signals from two or more such discovery tools emanating from the same multi-tool hand-held device may be interlaced or superimposed, (mapped,) using a computer to reveal more information about the focus area as compared to a single discovery tool. Those skilled in the art will readily appreciate the utility of such interlacing and mapping techniques which may be accomplished by various methodologies known in the art.

In certain embodiments of the present invention, the multi-purpose hand-held device is designed to accept and manipulate a physician's pre-owned viewing apparatus (endoscope).

In certain embodiments of the present invention, the viewing device 118 (as shown in FIG. 1) comprises various visible and non-visible wavelength transmitters and sensors for detecting any hot spots caused by cancer, infections, and the like. Further the viewing device 118 may be used as a sensor for measuring body's temperatures.

In an alternate embodiment, the use of two multi-purpose hand-held devices creates a stereoscopic view when both tools are used in proximity to one another. This can be accomplished by registering both images captured at different angles via a processor and then processing each image with different hues to be decoded via special 3D type glasses, or other 3D techniques know in the art. This technique would be particularly useful for both teaching and remote robotic procedures.

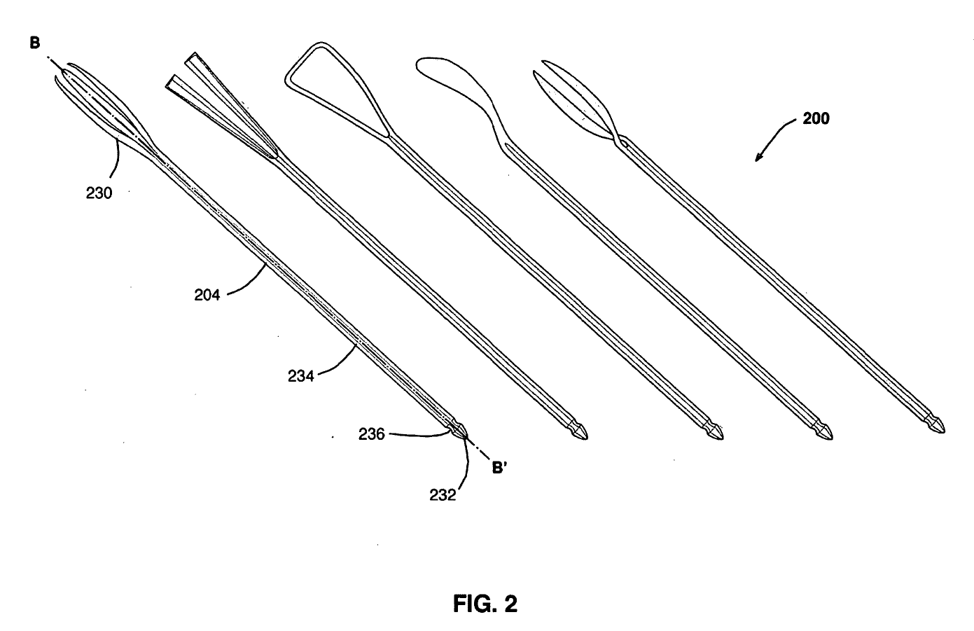

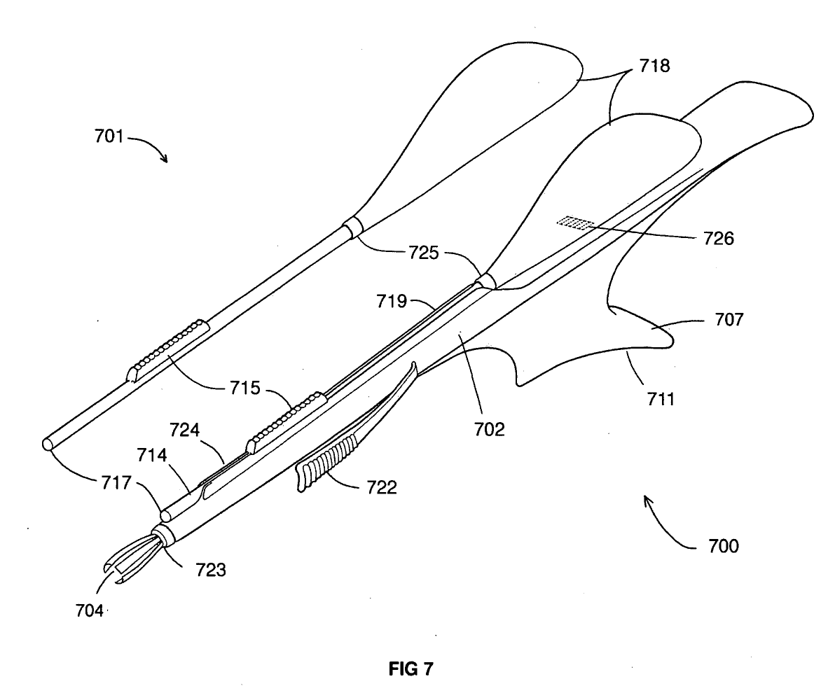

FIG. 2 illustrates a set of sample bits 200 according to the various embodiments of the present invention as shown in FIG. 1 at 104 and FIG. 7 at 704. The bit 204, as used herein, refers to any bit from the set of bits 200. By way of example and by no way of limitation, the bit 204 has been used herein for the purposes of explanation. The use of other similar bits is readily obvious to persons of ordinary skill in the art. The bit 204 can comprise a tool end 230, a receiving end 232, a shaft 234, and a notch 236. The bit 204 defines a longitudinal axis B-B′, as shown in the figure. The bit 204 is received by the multi-purpose hand-held device 102 as shown in FIG. 1 or 704 as shown in FIG. 7. When the multi-purpose hand-held device is used as an ear cleaning instrument, the tool end 230 may be used for removing wax and cleaning the external auditory canal. The tool end 230 may have shapes similar to a spoon, scoop, tweezers, threezer, scorp, open spoon, grabber, cutter, nipper, scissors and scalpel among others. Further, any one bit from the bits 200 may, for example, be rapidly used interchangeably with the multi-purpose hand-held device 102 or 702. Bits with different tool ends facilitate a varied number of operations in the external auditory canal depending on the requirement of the procedure. When the multi-purpose hand-held device is used as an ear cleaning device, a tweezing type tool end is used to grab harder, more solid wax; whereas, other tool ends are used to handle softer types of wax in such an application. In one embodiment, the cross-section of the shaft 234 has a hexagonal geometry with a tapered receiving end 232, while in other embodiments, the cross-section of the shaft 234 may have other geometries such as an oval or other-sided polygons. The shaft 234 is solid in one embodiment, while in another embodiment, at least a portion of the shaft 234 is hollow. Further, in an alternative embodiment, the tool end 230 is detachable from the shaft 234. The receiving end 232 can be tapered to a pointed tip just past a notch 236. Such a notch 236 can facilitate releasable holding of the bit 204 within the multi-purpose hand-held device 102 or 702. However, as will be apparent to those skilled in the art, the bit 204 may be configured in various ways known such that the bit 204 is receivable by the multi-purpose hand-held device 102 or 702.

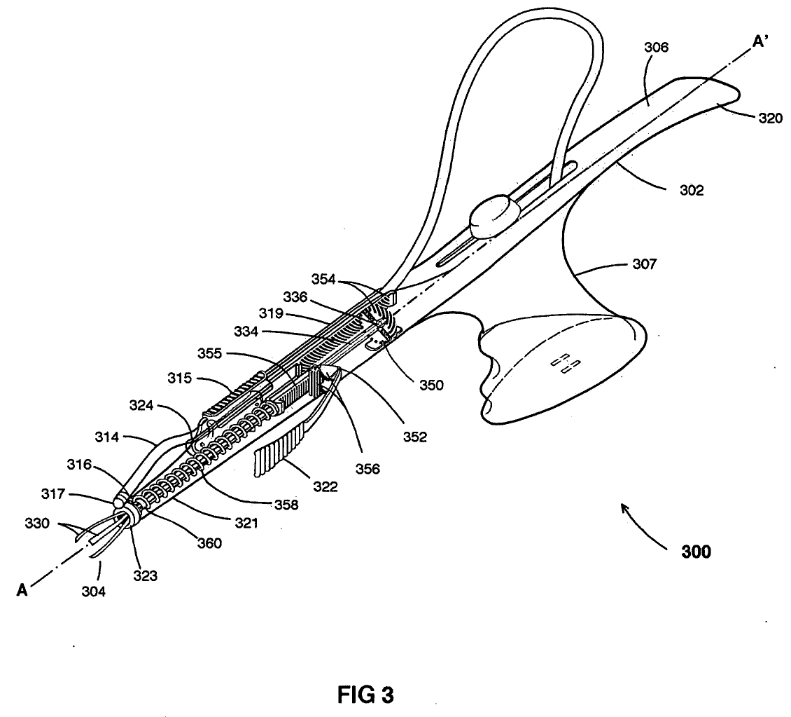

FIG. 3 illustrates an assembly 300 showing interior structure and mechanics of the multi-purpose hand-held device 302 and a bit 304. The multi-purpose hand-held device 302 further comprises a handle 307, viewing means actuator 318, view controlling means actuator 315, a rotary controlling means actuator 324 (far side ghosted in), a pincing controlling means actuator 322, a pincing controller hinge 352, a chuck 350, a worm rack gear system 354, a rack, a pinion gear system 356, a constriction collar 360, a return spring 358, and a removable light lens 323 that is releasably attached (via screw threads or other,) to the supporting body 306 and disposed at the distal end 321. The removable light lens tip 323 retains the compression type return spring and may be removed to dismantle the various parts for replacement and cleaning of the multi-purpose hand-held device 302. The chuck 350 is disposed in between the proximal end 320 and the distal end 321. When the bit 304 is received by the multi-purpose hand-held device 302, the receiving end of the bit 304 is releasably coupled both along axis A-A′ and torsionally to the chuck 350. The various elements of the multi-purpose hand-held device 302 are configured to control view, pincing, grabbing, cutting and rotation of the bit 304.

Further in an alternate embodiment, the rotational and pincing movement of the bit can be operated with a single double action controller and the use of just one finger or thumb. For example, pressing down on double action controller could cause the pincing motion and pulling back on the same double action controller could cause the rotation of the bit or vice a versa. Mechanical connections of the double action controller to the bit may be accomplished by various methodologies known in art.

The elements of the multi-purpose hand-held device 302 have been used herein to provide an example and by no way limit the scope of the present invention. As will be apparent to those skilled in the art, the present embodiment of the multi-purpose hand-held device 302 has been used herein for the purposes of explanation of the working of the multi-purpose hand-held device 302, and the use of other equivalent elements in the multi-purpose hand-held device is readily obvious to persons of ordinary skill in the art.

In the current embodiment (FIG. 3) the view controlling means actuator 315 is operably coupled to the supporting body 306 and the fiber element 314 of the viewing device controlling the angle of the distal lens 317. The view controlling means actuator 315 is disposed near the distal end 321. In certain embodiments, the view controlling means actuator 315 may be disposed near the proximal end 320. Further, in certain other embodiments, the view controlling means actuator 315 can be repositioned anywhere along the view control channel 319 by holding the fiber element 314 or the fiber cable near viewing means 318 firmly while sliding the view control means actuator 315 to a position preferred by the user. Because the distal end of the viewing device is pinned at hinge 316, it can bow the viewing device 314 in a vertical direction which causes the lens 317 to tilt up and down for a flexible view. In certain embodiments, the view controlling means actuator 315 provides finger manipulatable controls of the viewing device 314 using fingers and/or thumbs of left or right hand.

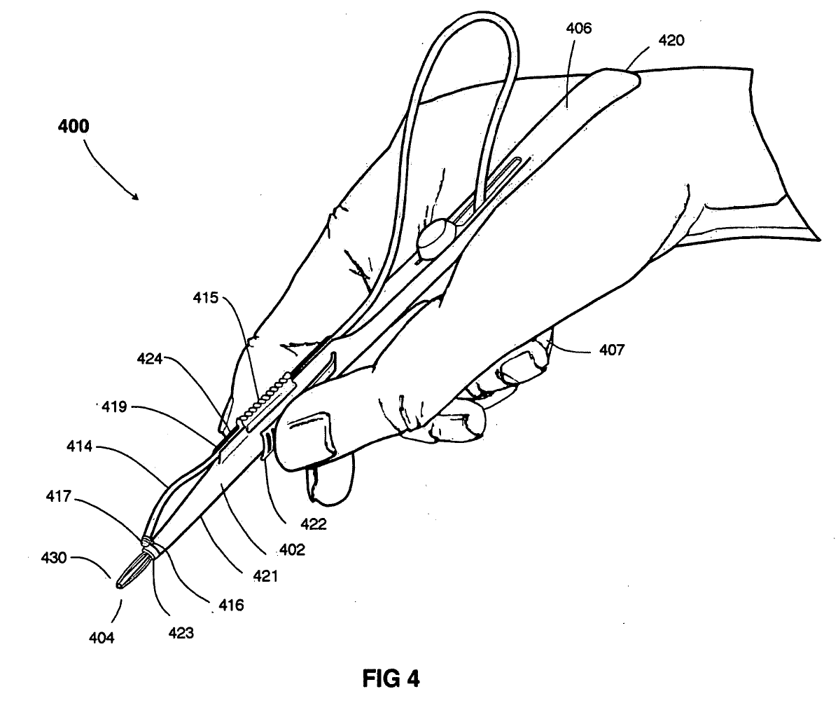

The view controlling means actuator 315, as shown in FIG. 4 as 415 is being controlled using the index finger of the right hand, but the view controlling means actuator 315 may be configured to be controlled by any of the fingers or thumbs of the left or right hand. in an alternate embodiment, the rotational and pincing movement of the bit can be operated with a single double action controller and the use of just one finger or thumb. The shape and position of the view controlling means actuator 315 can also be altered from what is shown without changing the spirit or scope of the invention.

The rotary controlling means actuator 324 (far side of the tool—ghosted in) is coupled to the supporting body 306 and controls rotation of the bit 304. The rotary controlling means actuator 324 can be disposed near the distal end 321. Further, in certain other embodiments, the rotary controlling means actuator 324 is disposed anywhere along the front part of the tool 321. The rotary controlling means actuator 324 is designed long in length to accommodate various relative length of fingers in different hands. The rotary controlling means actuator 324 can be controlled, for example, using the index finger of the right or left hand. According to certain embodiments of the present invention, the rotary controlling means actuator 324 is coupled to the chuck 350 through the worm rack gear system 354. The chuck 350 releasably holds the bit 304, for example through a set of springs and ball bearings. The worm rack system 354 converts linear force exerted onto the rotary controlling means actuator 324 into torque acting on the chuck 350. The cross-sectional shape of the bit shaft 234 couples with the bit opening in the chuck 350 providing proportional continuity between the chuck 350 and the bit 304. Therefore, when force is applied to the rotary controlling means actuator 324, the chuck 350 rotates depending on the direction of the force and consequently the chuck 350 rotates the bit 304. As will be apparent to those skilled in the art, the bit 304 may be coupled to the rotary controlling means actuator 324 using various techniques known in the art. The bit 304 is coupled to the rotary controlling means actuator 324 such that rotating controlling means actuator 324 is configured to rotate the bit 304. The rotary controlling means actuator 324 is pressed using any finger or thumb on either hand to rotate the bit 304. The bit 304 is rotated and adjusted according to the need or to the convenience of the user to achieve desired functionality for operating within a body cavity. The rotary controlling means actuator 324 extends forearm twisting motions to the dexterity of fingers and thumbs of the left or right hand.

As will be apparent to those skilled in the art, the rotating chuck 350 and bit 304 may be coupled to the rotary controlling means actuator 324 using various techniques known in the art. Alternatively, the rotary controlling means actuator 324 may be controlled using any of the fingers of the left or right hand. In another embodiment, the rotary controlling means actuator 324 may be controlled using the thumb of the left or right hand. The shape and position of the rotation controlling means actuator can also be altered from what is shown without changing the spirit or scope of the invention.

The pincing controlling means actuator 322 is coupled to the supporting body 306 and controls pincing movement of the bit 304 by alternately opening and closing the tines 330 of the bit 304. The pincing controlling means actuator 322 is disposed near the distal end 321 substantially opposite the rotary controlling means actuator 324. The pincing controlling means actuator 322 is shown designed long in length to accommodate various relative lengths of fingers in different hands. According to certain embodiments of the present invention, the pincing controlling means actuator 322 is connected to the rack 355 through the reversing rack and pinion gear system 356. The reversing rack and pinion gear system 356 converts pincing force of the fingers, via the pincing controlling means actuator 322, into lateral movement of the extension tube and rack 355 and constriction collar 360. Since the operable rack 355 is coupled to the constriction collar 360, force exerted onto the pincing controlling means actuator 322 is transformed into pincing movement of the bit when the constriction collar 360 is forced from the thinner section of the shaft 334 towards the base of the bit tines 330 (proximal end of the bit tines 330) which is increasingly wider than the collar as it moves towards the distal end of the bit tines 330. Since the collar 360 does not expand, the bit tines 330 must constrict. Therefore, when the force is applied to the pincing controlling means actuator 322 the constriction collar 360 moves laterally forward (towards the distal end of the bit 304) along the axis A-A,′ and the bit closes. The distal end of the concentrically-loaded compression type return spring 358 can bear on a ledge of the removable light lens tip 323 (distal end) and on the butt of the rack 355. The spring 358 can, in this embodiment, push the constriction collar 360, the rack and pinion gear system and the pincing controlling means actuator 322 back to its original position when force of the fingers is removed from the pincing controlling means actuator 322. The pincing controlling means actuator 322 is thus configured to move the constriction collar 360 longitudinally, along axis A-A′, towards the distal end of the bit 304 thereby acting on the bit tines 330 such as nipper, cutter, grabber scissors, and the like. The bit tines 330 of the bit 304 thus may be operably opened and closed when alternately compressed and decompressed by the fingers. The pincing controlling means actuator 322 thus extends the dexterity of fingers and thumbs of the left or right hand without any impedance to the other controls of the multi-purpose hand-held device 302, simulating the natural picking an grabbing and cutting motions of the thumb and index finger with various shaped bits in areas, (blind cavities) otherwise inaccessible to the girth of fingers.

As will be apparent to those skilled in the art, the constriction collar 360 may be coupled to the pincing controlling means actuator 322 using various techniques known in the art. The pincing controlling means actuator 322 is shown in FIG. 4 being controlled by the thumb of the right hand. Alternatively, the pincing controlling means actuator 322 may be controlled using any of the fingers or thumbs of the left or right hand. The shape and position of the pincing controlling means actuator 322 can also be altered from what is shown without changing the spirit or scope of the invention.

FIG. 4 illustrates an assembly 400 of the multi-purpose hand-held device 402 and a bit 404 according to various embodiments of the present invention. The multi-purpose hand-held device 402 is held in the palm region of the hand between the thumb and the index finger. The bit 404 is placed into the multi-purpose hand-held device 402 for example by inserting the notch end of the bit 232 (as shown in FIG. 2) of the bit 404 into the removable light tip opening 423. The bit 404 is inserted into the multi-purpose hand-held device 402 until the bit 404 is snapped into the opening of the chuck 350 (as shown in FIG. 3 and described above.) As shown in the embodiment of FIG. 4, the thumb controls the pincing movement of the bit tines 430 using a pincing controlling means actuator 422. The index finger controls the rotation of the bit 404 using a rotary controlling means actuator 424 (far side, not seen.) A viewing device 414 may be controlled though view controlling means actuator 415 using either the index finger or thumb. The multi-purpose hand-held device 402 may be held using the left or right hand according to the convenience, comfort and dexterity of the user. In one aspect, the present invention provides a method of using the assembly 400. The method comprises controlling movement of the viewing device 414, controlling rotational movement of the bit 404, and controlling pincing movement of the bit 404.

The multi-purpose hand-held device 402 combines the basic functions of fingers, hand, wrist, and eyes miniaturized into a functional single handed device. The fingers or thumb of the left or right hand controls movements of the bit 404 inside the cavity from a vantage remote from the body cavity. The fingers or thumb of either left or right hand allow smooth controlled use of the multi-purpose hand-held device 402. The dexterity can be imparted, in some embodiments, by configuring the controls on the multi-purpose hand-held device 402 such that any of the fingers or the thumbs on either hand may control any of the view controlling means actuator 415, the rotary controlling means actuator 424, and the pincing controlling means actuator 422. Further, the smooth operation is enhanced by controlling any of the above mentioned controlling means actuator (view controlling means actuator 415, the rotary controlling means actuator 424, and the pincing controlling means actuator 422) individually or simultaneously. Further, each function is controlled by a single finger which allows the multi-purpose hand-held device 400 to be easily operated by simple finger control within any body cavity.

The view controlling means actuator 415, the rotary controlling means actuator 424 and the pincing controlling means actuator 422 is shown in FIG. 4 being controlled by the index finger and thumb of the right hand with the handle 407 in the right hand. Alternatively, embodiments can be configured or adjusted such that the various controlling means may be controlled using any of the fingers of the left hand or the right hand. The shape and position of the various controlling means can also be altered from what is shown without changing the spirit or scope of the invention.

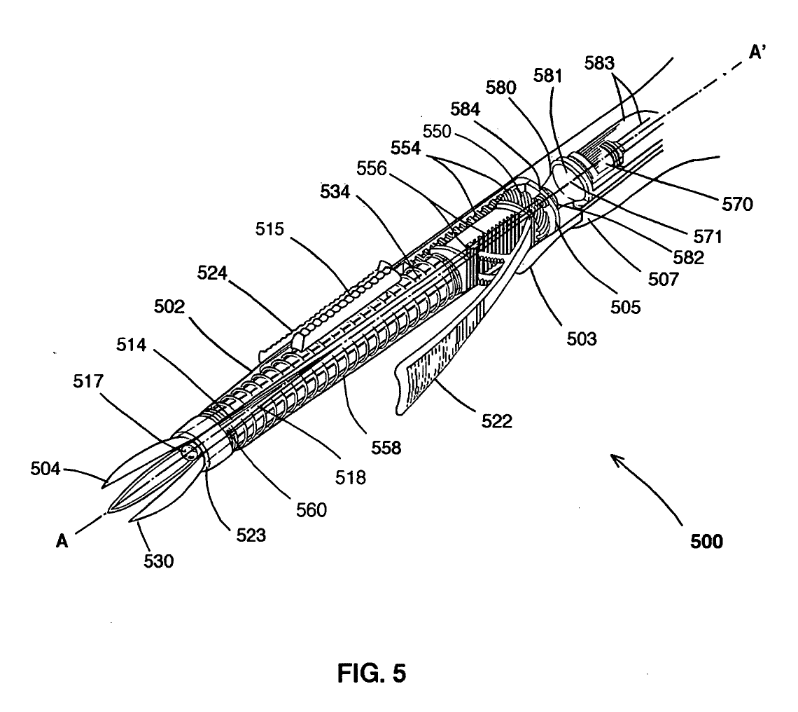

FIG. 5 illustrates an assembly 500 showing the interior structure and mechanics of the multi-purpose hand-held device 502 and a bit 504. The multi-purpose hand-held device 500 comprises a generally tubular body 502, a rotary controlling means actuator 524, a rack and worm gear system 554, a pincing controlling means actuator 522, a rack and pinion system 556, an extension tube and constriction collar 560, a return compression type spring 558, a view controlling means actuator 515, a centrally located hard (glass shaft) camera 518, a micro lens 517 a removable light ring 523, a camera housing 580, a detachable connector seat 584, that is releasable and connects to the body via the locking hardware 505, an operable camera release and retractor lever 503 located at the front top of the handle 507, that allows for the retraction of the camera prior to insertion and extraction of the hollow core hexagonal section bits 504, to prevent damage to the delicate camera parts, a stainless steel or other encased glass optical shaft 514, operable bit tines 530, a chuck 550 that releases and accepts bits only when the camera is properly retracted, a concave digital sensor 581, with a centrally located pin hole for laser projection 571, a mid correction lens or set of lenses 582, a laser generator 570, and bus cabling to the various power sources, transmitter, and processors 583.

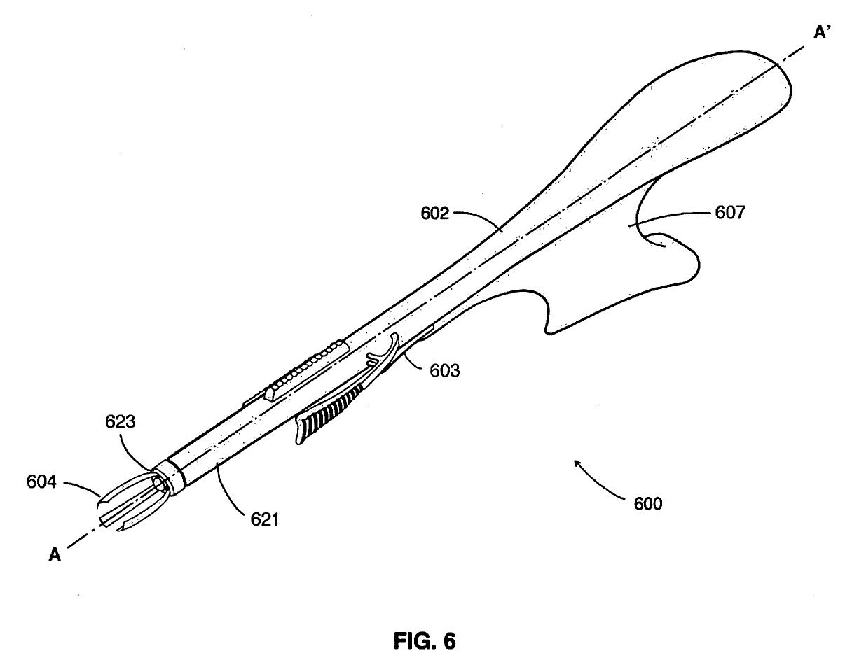

According to one embodiment shown in FIGS. 5 and 6, bits used with the apparatus 502 may be modified to accommodate alternative viewing devices. In this embodiment, the shaft 534 of the bit 504 is hollow and provides a location for the viewing device 518. The viewing device 518 is configured to be concentrically loaded inside the bit 504 such that the alternative viewing device 518 does not interfere with the coupling of the bit 504 to the chuck 550 and the operation of the bit 504. This is accomplished by locating the alternative viewing device 518 near the chuck 550. Further, the hollow shaft 534 accommodates a hard camera such as a glass optic 514 that is coupled to the viewing device 518 near the chuck 550, of which the distal glass optic emerges in the vicinity of the distal end of the bit times. The rigid casing of the glass optical shaft 514 provides a secure environment to the glass optic which advantageously can provide for generally higher quality images.

The multi-purpose hand-held device assembly 600 comprises a supporting body 602, a handle 607, and internal viewing device (not shown, see 514). The supporting body 602 is a generally tubular body configured to receive the bit 604. Examples of the supporting body 602 may include metallic frames, non-metallic frames, and the like. The supporting body 602 comprises a distal end 621 and a proximal end 620. The supporting body 602 defines a body axis A-A′ as shown in the figure. When the multi-purpose hand-held device 602 is held before a body cavity, the distal end is placed near or into the cavity. The bit 604 is received by the distal end 621 at the opening of the removable light tip 623. The handle 607 is attached to the supporting body 602 near the proximal end. In the illustrated embodiment, the handle 607 along with the sensitive camera parts is detachable from the supporting body 602. The handle 607 can, for example, contain and retain the sensitive components, such as components of the imaging means, data handling, data transmission, control or other electronics, or the like and may be removed for autoclave cleaning of the remainder parts of the multi-purpose hand-held device 602 by operating lever 603. Further, the handle 607 may be replaced by handles of varied shapes and sizes to adjust to palm size of the user such as a nurse or a doctor. In other embodiments, the supporting body 602 and the handle 607 are configured such that the handle 607 may slide relative to the supporting body 602. The handle 607 is movable toward or away from the proximal end. Therefore, the handle 607 may be quickly adjusted according to the comfort or size of the palm of the user, such as a nurse or a doctor. In alternative embodiments, the handle 607 may be rotated around the girth of the supporting body 602 for added comfort of the user and/or depending upon the application of the assembly 600. The handle 607 easily adjusts to fit fingers of different length and sliding the handle 607 towards the distal end or towards the proximal end.

FIG. 6 illustrates an assembly 600 according to various embodiments of the present invention. The assembly 600 comprises a multi-purpose hand-held device 602 and a bit 604. In one embodiment the tool is configured to be used for seeing and operating within a body cavity such as the ear canal. In alternative embodiments, the bit 604 may be inserted through an opening (not shown in the figures) made into the supporting body 602, parallel to the body axis A-A′. The multi-purpose hand-held device 602 may be held in the right or left hand and the various controlling means can be operated by any finger or thumb on either hand. The bit 604 is received by the multi-purpose hand-held device 602, and the bit 604 is configured to operate within a body cavity.

Further, in another embodiment, the handle 607 comprises power cells and a transmitter unit (not shown) placed inside the handle 607. The power cells may be rechargeable power cells. The multi-purpose hand-held device 602 may be recharged by connecting the rechargeable power cells of the handle 607 into a detachable recharging base or plugged into a wall outlet via a transformer to run while charging is taking place. The transmitter unit may be connected to a display device through a wire or wirelessly.

FIG. 7 is a perspective view of an alternate embodiment, assembly 700, of a removable, offset (not centrally located) hard camera with adjustable focal length. Assembly 700 is comprised of similar bit operations including a rotational controlling device 724 and a pincing controlling device 722. Assembly 701 shows the hard camera (endoscope) not inserted into the device body 702. In this embodiment a non-concentric removable hard camera (glass) 701 is slid into a view control channel 719 making electrical contacts at 726 (dashed in). The view controller 715 shifts the distal lens 717 and glass shaft 714 (distal end,) in relation to the lens/es and sensor at the camera (proximal) end of the viewing device 718 by changing the relative distance at slip joint 725 between lenses. The focal length changes giving this camera the ability to clearly focus the image at various distances. This removable viewing device 718 can for example contain all of the electronics for sensing wavelengths, transmitting images and data via Bluetooth or other technologies to a processor and powering itself via rechargeable cells with charging contacts at 711 on adjustable handle 707. A laser generator could also be incorporated into the viewing device 718 for the purpose of cauterizing or cutting. In addition, this removable hard camera can be inserted into other tools that combine vision and manipulation that are built to accept it. The removable light ring 723 as shown in this embodiment shines out of the tip only, preventing any light, not bouncing off the subject first, from reducing the image quality captured at the lens 717. In another embodiment, not shown in FIG. 7, is where the viewing device 718 is a flexible camera like a fiber optic or like (endoscope).

FIG. 8 is a perspective view of an embodiment of the assembly 800 which is comprised of a processor, a power assisted boom and an adjustable monitor which may be used with the multi-purpose hand-held device such as shown in FIGS. 1, 3, 4, 5, 6, and 7 and described herein. Assembly 800 processes and projects images captured by the camera onto a movable monitor. The processor and electronics as shown are located in the ceiling mount 890 but can be located elsewhere as known in the art. Small servo-motors at each joint 893 respond to movements of the multi-directional switches 894 located at each lower corner of the monitor 892 to allow subtle repositioning of the monitor 892 with the one remaining hand of the user while a procedure is in process. More specifically the multi-directional switches 894 send signals to the processor located in ceiling mount 890 which coordinate multiple motors to produce the desired movement. The user needs only to move one of the multi-directional switches 894 (left or right handed) in the intended direction to reposition the monitor 892 as needed. The positioning of the monitor 892 adjacent to the site of the procedure and the ease of repositioning the monitor 892 with one hand, as needed, is critical in delicate situations, like ear cleaning of small children, since both the doctor and the patient need to, and do shift often. The half dozen or so dials 895 located at the bottom edge of the monitor 892 can be attached for the procedure by the physician to various frequency and level controls at the processor so that the view is enhanced for greater discovery. The boom arm 891 attaches to both the ceiling mount 890 and the monitor 892. In another embodiment not shown in FIG. 8, the boom arm 891 and joints 893 would operate manually without motors.

The present invention can also utilize wire or wirelessly connected input devices such as keyboard and mouse or other input devices as known to those in the art.

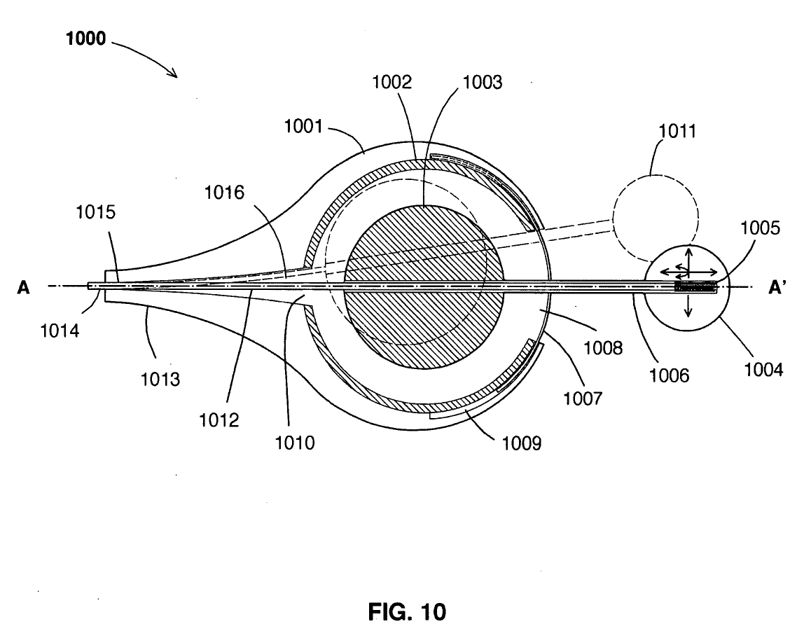

FIG. 10 shows a detail section of a omni-directional-multi-speed switch assembly 1000 (see 894) that can be used to move the power assisted monitor boom arm in any direction at various speeds with one controller means actuator and the one remaining hand (i.e., the hand not using the tool). The switch consists of a body 1001, a controller 1004 located at the proximal end of the switch, a spherical cathode 1003 centered inside of a larger spherical anode or sensor 1002, a flexible shaft 1012 which is concentrically loaded and partially enveloped by an outer shaft 1006 that connects the cathode 1003 to the handle 1004. A semi-spherical cover 1007 covers the opening 1008 which allows free movement of the outer shaft 1006. The cover 1007 slides freely in a similarly shaped opening 1009. A second opening 1010 at the distal end of the outer sphere 1002 and a conical opening 1016 further towards the distal end, allows free movement of the flexible shaft 1012. The distal end of the flexible shaft 1012 can be rigidly connected to the switch body 1015 at the distal end of the switch 1013. Electrical contacts for both the cathode and anode are at 1014. (Electrical bias can be reversed as appropriate.) The flexible shaft 1012 can be connected to the controller 1004 via a tension/compression type spring allowing the controller 1004, the outer shaft 1006 and the cathode 1003 to operate in either direction along the axis A-A′. The cathode 1003 is at rest centered inside the cavity of the anode 1002. Because of the dual spring action and the 360 degree flexible shaft 1012 the controller 1004 can move the spherical cathode towards any inside surface of the spherical anode 1002 (see position 1011 dotted in). The signal generated by the proximity of the point of tangency of the cathode 1003 to the anode 1002 is transferred to the processor where it is interpreted as a ray or a direction of movement. As well as determining intended direction of movement, the switch also senses the intended speed of movement. The closer the cathode gets to the anode the stronger the signal that is transferred to the processor—which is interpreted as variable speed. In use, the movement of the controller 1004 in any direction with variable force will be processed for like movement of the boom arm by servo-motors located at each joint of the arm.

In an alternate embodiment the anode may be an approximation of a sphere to maintain a consistent distance between the cathode and anode surfaces as the cathode is moved through the extent of its zone of movement. In a further embodiment the handle may be of alternate shapes.

In another embodiment, the multi-purpose hand-held device as shown in FIGS. 1, 3, 4, 5, 6, and 7 and described herein may be used with a display device known in the art and not the display device describe above and depicted in FIG. 8.

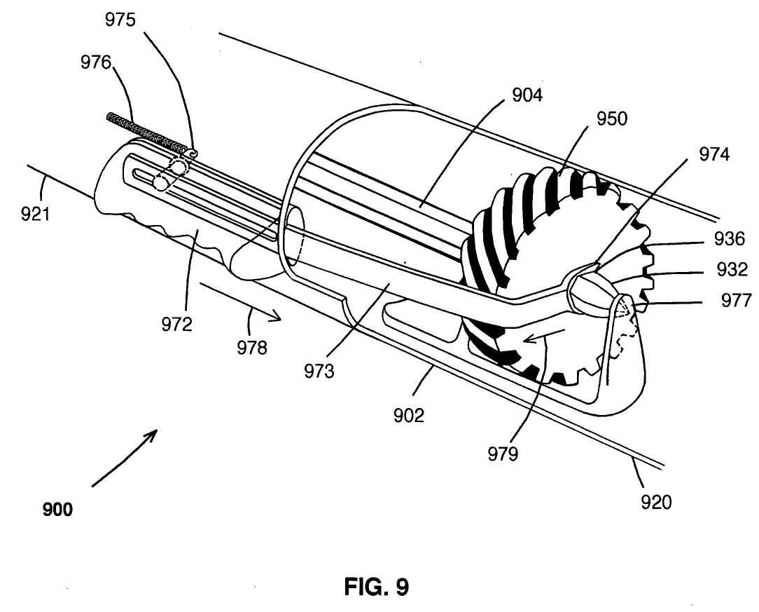

FIG. 9 is alternative quick change mechanism 900 for operably and releasably retaining the bit which may be used in lieu of the bit mechanism as shown in FIGS. 1, 3, 4, 5, 6, and 7 and described herein. Note that for clarity the drawing has omitted some adjacent parts that are similar to previous embodiments but not pertinent to this description. The hexagonal (or other,) cross-sectional shape of the shaft 904 releasable couples with the same shape void at the center of the chuck 950 so that when the chuck 950 is turned by the worm rack (not shown,) the bit 904 turns in a torsional manner. A bit is loaded in two methods. The first method is accomplished by inserting the bit into the opening at the distal end of the multi-purpose hand-held device 902 until it snaps into place. The retaining clip 974 springs out of the way in the direction shown by arrow 979 due to the geometries of both the retaining clip 974 and the receiving end 932 in a manner known to those in the art. The bit is longitudinally (in the axis of the bit) retained by the retaining clip 974 which couples with reglet 936 cut into the circumference of the shaft of the bit, near the receiving end 932 (proximal end.) The retaining clip 974 retains the bit in a longitudinal manner while allowing free torsional movement of the bit as the chuck 950 is turned. Releasing the bit is accomplished by sliding the bit lock controller 972 in the direction of the arrow 978 towards the proximal end 920 of the device. The movement of the bit lock controller 972 constrains the spring loaded clip mechanism 973 towards the inside of the body of the multi-purpose hand-held device 902 pulling it out of the reglet 936 on the bit allowing the bit to slide freely out of the chuck 950 and the body of the multi-purpose hand-held device 902. The second method of inserting the bit 934 allows for a method that eliminates the need to touch any sharp tip of any bit to load the tool. By holding the bit lock controller 972 in the load position (in the direction of arrow 978 towards the proximal end of the device 920,) the bit can be dropped into the opening of the distal end of the multi-purpose hand-held device 902 and allowed to slide by gravity until it hits the bit stop 977. At this point the bit lock controller 972 can be released. The tension type return spring 976 which is operably connected to both the bit lock controller 972 at 975 and the body of the multi-purpose hand-held device 902 (not shown,) causes the spring action of the arm of the retaining clip 973 to engage the retaining clip 974 into the bit reglet 936 to operably retain the bit for use. (For the purpose of this application a reglet is an indent or slot that can be engaged to retain the bit.)

The quick change mechanism 900 allows one to insert a bit with a sharp blade (for example) without having to apply pressure near the blade sufficient to activate a snap-in retainer or without having to grip the sharp end of the bit to load it into the device. In some embodiments, the option to use the snap-in retainer to insert a bit remains.

As will be apparent to those skilled in the art, the multi-purpose hand held device and multi-purpose tool may be used for various applications. A non-exhaustive list of such applications includes surgery, general medicine, micro surgery, electronics, robotics, micro-robotic-surgery hands, otoscope replacement and the like. By providing micro motors and sensors the multi-purpose hand-held device may be used for remote operations in micro surgery.

The embodiments of the present invention, as apparent from the discussion above, provide for various advantages. In one aspect, the present invention provides an improved multi-purpose hand-held device easy to be handled using left or right hand.

In another aspect, in certain embodiments, the multi-purpose hand-held device is provided which extends the natural movement and dexterity of index finger and thumbs without any impendency in using the multi-purpose hand-held device.

Further, the present invention provides, in certain embodiments, a viewing device to see the interior of a blind cavity, such as the auditory canal, while performing the cleaning operation rather than the state of the art with one tool for looking into the canal and second tool for cleaning the canal.

Further in another aspect, in certain embodiments, the multi-purpose hand-held device is provided which performs the function of seeing simultaneously while operating inside a body cavity.

Furthermore, in another aspect, in certain embodiments, LASER technology may be provided for various techniques of reading the surface temperature, depth gauge, or even as a cutting, excavation or extraction device.

Furthermore, in another aspect, in certain embodiments, the multi-purpose tool is easy, safe, and efficient to use.

U.S. Provisional Application 61/008,371, filed 20 Dec. 2007, to which this application claims priority, is hereby incorporated by reference in its entirety.

While the foregoing is directed to embodiments of the present invention, other and further embodiments of the invention may be devised without departing from the basic scope thereof, and the scope thereof is determined by the claims that follow.

1. A multi-purpose tool comprising:
a supporting body adapted to receive and support a bit;
a handle attached to the supporting body;
a view controlling means coupled to, or adapted to couple to, the supporting body for controlling the view of a viewing device; and
one or more of the following control means:
a rotatory controlling means coupled to the supporting body for controlling the rotational movement of a bit; and
a pincer controlling means coupled to the supporting body for controlling pincing movement of the bit.

2. The multi-purpose tool of claim 1, further comprising a viewing device coupled to, or adapted to couple to, the supporting body in a manner functionally engaged with the view controlling means.

3. The multi-purpose tool of claim 1, wherein the viewing device comprises a glass optic.

4. The multi-purpose tool of claim 3, wherein the supporting body is adapted to receive a hollow bit with a tool end, and wherein the a glass optic is shaped and positioned or adapted to position so that the hollow bit can be slipped over the glass optic to be received by the supporting body, such that the glass optic is positioned to visualize one or more areas that project along approximately a center axis of the tool bit.

5. The multi-purpose tool of claim 1 comprising:
the rotatory controlling means coupled to the supporting body for controlling the rotational movement of a bit.

6. The multi-purpose tool of claim 5, wherein the view controlling means controls angle of the viewing device.

7. The multi-purpose tool of claim 5, wherein the viewing device is configured to capture and provide two or more interlaced wavelengths.

8. The multi-purpose tool of claim 5, wherein the handle is adjustable to provide variable positions of the handle relative to the supporting body.

9. The multi-purpose tool of claim 5, wherein the handle is removable.

10. The multi-purpose tool of claim 9, wherein the tool is configured for autoclave cleaning after removing the handle.

11. The multi-purpose tool of claim 1 comprising:
the pincer controlling means coupled to, or adapted to couple to, the supporting body for controlling pincing movement of the bit.

12. The multi-purpose tool of claim 11, wherein the view controlling means controls angle of the viewing device.

13. The multi-purpose tool of claim 11, wherein the viewing device is configured to capture and provide two or more interlaced wavelengths.

14. The multi-purpose tool of claim 11, wherein the handle is adjustable to provide variable positions of the handle relative to the supporting body.

15. The multi-purpose tool of claim 11, wherein the handle is removable.

16. The multi-purpose tool of claim 15, wherein the tool is configured for autoclave cleaning after removing the handle.

17. The multi-purpose tool of claim 1 comprising:
the rotatory controlling means coupled to the supporting body for controlling the rotational movement of a bit; and
the pincer controlling means coupled to, or adapted to couple to, the supporting body for controlling pincing movement of the bit.

18. The multi-purpose tool of claim 17, wherein the view controlling means controls angle of the viewing device.

19. The multi-purpose tool of claim 17, wherein the viewing device is configured to capture and provide two or more wavelengths for interlacing.

20. The multi-purpose tool of claim 17, wherein the handle is adjustable to provide variable positions relative to the supporting body.

21. The multi-purpose tool of claim 17, wherein the handle is removable.

22. The multi-purpose tool of claim 21, wherein the tool is configured for autoclave cleaning after removing the handle.

23. A multi-purpose tool comprising:
a supporting body adapted to receive and support a bit;
a handle attached to the supporting body;
a rotatory controlling means coupled to the supporting body for controlling the rotational movement of a bit; and
a pincer controlling means coupled to the supporting body for controlling pincing movement of the bit, and wherein one of the following pertains:
(i) the supporting body is adapted to receive and support a viewing device; or
(ii) the tool further comprises a viewing device.

24. A method of a user operating a multi-purpose tool of claim 1, comprising providing the tool;
providing a power-assisted monitor comprising a means for moving the monitor with one hand; and
operating the monitor with one hand and using the other hand to operate the tool by one or more of adjusting the view controlling means or the rotatory controlling means.

25. The method of claim 24, wherein operating the monitor comprises one or more of (a) adjusting via controls on the monitor light intensity provided by the tool, (b) adjusting via controls on the monitor wavelength of the provided light, or (c) adjusting via controls on the monitor the processing of camera output from the tool.

26. The method of claim 24, wherein operating the monitor comprises moving the monitor utilizing the means for moving.

27. A method of a user operating a multi-purpose tool of claim 5, comprising providing the tool;
providing a power-assisted monitor comprising a means for moving the monitor with one hand; and
operating the monitor with one hand and using the other hand to operate the tool by one or more of adjusting the view controlling means or the rotatory controlling means.

28. A method of a user operating a multi-purpose tool of claim 11, comprising providing the tool;
providing a power-assisted monitor comprising a means for moving the monitor with one hand; and
operating the monitor with one hand and using the other hand to operate the tool by one or more of adjusting the view controlling means or the pincing movement controlling means.

29. A method of a user operating a multi-purpose tool of claim 17, comprising providing the tool;
providing a power-assisted monitor comprising a means for moving the monitor with one hand; and
operating the monitor with one hand and using the other hand to operate the tool by one or more of adjusting the view controlling means or the rotatory controlling means or the pincing controlling means.

30. The multi-purpose tool of claim 1, further comprising:
a retaining mechanism for retaining a bit having a reglet, the mechanism comprising clip adapted to engage the reglet that is spring biased to the position for engaging the reglet but where the clip can be levered out of the engagement position.

31. A multi-purpose tool comprising:
a supporting body adapted to receive and support a bit;
a handle attached to the supporting body;
a retaining mechanism for retaining a bit having a reglet, the mechanism comprising clip adapted to engage the reglet that is spring biased to the position for engaging the reglet but where the clip can be levered out of the engagement position.
